(19) United States
(12) Patent Application Publication
(10) Pub. No.: US 2025/0129001 A1
(43) Pub. Date: Apr. 24, 2025

(54) METHANOL PRODUCTION FROM BIOMASS AND GREEN HYDROGEN

(71) Applicant: FEV Group GmbH, Aachen (DE)

(72) Inventor: Thorsten SCHNORBUS, Winterberg (DE)

(73) Assignee: FEV Group GmbH, Aachen (DE)

(21) Appl. No.: 18/999,482

(22) Filed: Dec. 23, 2024

Related U.S. Application Data
(63) Continuation of application No. PCT/DE2023/100473, filed on Jun. 22, 2023.

(30) Foreign Application Priority Data
Jun. 27, 2022 (DE) 102022115977.9

(51) Int. Cl. C07C 29/152; B01J 19/00; B01J 19/24; C25B 1/04

(52) U.S. Cl. CPC C07C 29/152; B01J 19/0006; B01J 19/24; B01J 2219/00198; C25B 1/04

(57) ABSTRACT

In a process for producing methanol, a synthesis gas that has been recovered from biomass is fed to a methanol synthesis apparatus. In a main operating mode in which sufficient electrical power is available for electrolytic hydrogen recovery, correspondingly electrolytically recovered hydrogen is fed to the methanol synthesis apparatus. In a secondary operating mode in which insufficient electrical power is available for electrolytic production of hydrogen, a tail gas that arises from a biogas recovered from a biomass on removal of the synthesis gas is fed to a generator in order to provide electrical power for apparatuses involved in the process.

### CROSS-REFERENCE TO RELATED APPLICATIONS

This application is a continuation of International Application No. PCT/DE2023/100473, filed on Jun. 22, 2023, which claims priority to and the benefit of DE 10 2022 115 977.9, filed on Jun. 27, 2022. The disclosures of the above applications are incorporated herein by reference.

### FIELD

The present disclosure relates to a process for methanol synthesis and to a corresponding system.

### BACKGROUND

The statements in this section merely provide background information related to the present disclosure and may not constitute prior art.

EP 3 452 438 B1 discloses a process for producing biomethanol which is fed with electrolytically recovered hydrogen and organic waste materials. EP 3 017 015 B1 also discloses using steamcrackers in a first step of processing biomass, in order to divide a hydrocarbonaceous feedstock into shorter chains. The focus of such processes and apparatuses is on maximum efficiency of performance of the chemical transformations from feedstocks provided, such as biomass and hydrogen in particular. What is typically not considered here is that, in some cases, the feedstocks could be available in insufficient volumes for the transformations and/or that there can be fluctuations in the provision of the feedstocks in terms of volume or over time. Furthermore, demand for power not obtained by renewable means for the overall system of the methanol synthesis is to be reduced or avoided.

### SUMMARY

This section provides a general summary of the disclosure and is not a comprehensive disclosure of its full scope or all of its features.

The present invention is aimed at solving this problem. The features of the independent claims describe advantages of the invention. Preferred developments are the subject of the dependent claims.

In a process for producing methanol, a synthesis gas is recovered from biomass and is fed to a methanol synthesis apparatus. In this process, in a main operating mode in which sufficient electrical power is available for electrolytic hydrogen recovery, correspondingly electrolytically recovered hydrogen is fed to the methanol synthesis apparatus. And, in a secondary operating mode in which insufficient electrical power is available for electrolytic production of hydrogen, a tail gas that arises from a biogas recovered from a biomass on removal of the synthesis gas is fed to a generator in order to produce electrical power for apparatuses involved in the process. The tail gas is thus the difference between the biogas produced and the synthesis gas, which has especially been separated such that it is usable directly in the methanol synthesis apparatus. In the secondary operating mode, in particular, no electrolysis is conducted. The expression “power for electrolytic hydrogen recovery” especially means renewable power, for example from photovoltaics or wind power. In principle, however, other power sources, for example including from nuclear power, are also included.

Conventionally, a methanol synthesis apparatus is a unit that converts synthesis gas (H2 and CO) to methanol. In this particular case, however, rather than CO, it is predominantly CO₂ that is fed to the process. The synthesis unit works with a recirculation pump/fan that feeds the unconverted starting materials back to the synthesis. The synthesis forms methanol and water. Since small amounts of harmful/extrinsic gases or inert gases are also always fed to the process in the feed gases, it is necessary by means of a purge gas stream to prevent accumulation of these gases, in order not to impair the efficiency of the synthesis process. The process is exothermic, and the heat that arises can be used in other parts of the plant (for preheating of the distillation or the HyGas process). The outgoing gases in the purge gas stream can be utilized by various components of the system, such as, in particular, the generator or the oxyfuel burner.

Firstly, the volume of biogas cannot be varied efficiently over time in a bioreactor, especially in a HyGas generator, so as to result in a largely constant biogas stream over time. Secondly, the amount of photovoltaic power varies diurnally, and so there are resultant fluctuations in the diurnal/temporal ratio of these feedstocks for methanol synthesis. Because of the two above-described operating modes of the inventive process (combined with intermediate stages), it is possible at any time to obtain a maximum possible amount of methanol and simultaneously to reduce the required amount of nongreen power overall. Although the amount of methanol produced is distinctly reduced in the secondary operating mode, i.e. in the event of a lack of green power, compared to the main operating mode, supply of the residual gas from biomass utilization to the generator makes it nevertheless possible to conduct the synthesis without having to resort to nongreen power.

It is advantageous when, at least in the main operating mode, and preferably also in the secondary operating mode, a tail gas that arises from a biogas produced from a biomass on removal of the synthesis gas is fed to a processing operation in order thereby to produce further synthesis gas which is also fed to the methanol synthesis apparatus. The methanol synthesis apparatus requires simple chemical compounds, for example H2, CO₂, which are first separated from the biogas in a first step. In order to obtain a high yield of synthesis gas from the biogas, the residual constituents, i.e. the tail gas, are converted in the second step described such that it is usable as a “further synthesis gas”.

In the processing operation, it is especially advantageous when the tail gas is fed in a particular ratio to an oxyfuel burner and a reformer, wherein an oxidation is conducted in the oxyfuel burner and the oxidized gas is fed to the reformer, and the reformer conducts a processing operation with these gases, in particular a reduction, in order to be able to supply it as the further synthesis gas to the methanol synthesis apparatus. This processing operation can be used in the secondary operating mode too. An oxyfuel burner is especially a burner unit that burns hydrocarbons and other gases to generate heat. In this particular case, the gas is burnt with pure oxygen in order to avoid the introduction of, for example, nitrogen from the air. The combustion air ratio is substoichiometric in order to produce an oxygen-free offgas stream which can in turn be utilized as reactant in the further process steps. A reformer is thus a chemical unit that converts hydrocarbons to hydrogen and carbon dioxide/carbon monoxide. The process is endothermic and thus has to be supplied with heat from the outside.

It is also advantageous if the system can switch between the main operating mode and secondary operating mode depending on the available electrical green power or depending on the available hydrogen (the system is set up such that it is able to undertake this switching), or mixed forms of the operating modes are used, where the mixed forms differ, for example, in the ratio of the tail gas that is fed either to the generator or to the processing operation. A particularly suitable criterion for switchover (or the proportional adjustment or control) is the amount of available green power, since this is easy to measure. The amount of electrolytically recovered hydrogen is proportional to the green power, and so this electrolysis hydrogen is also usable as a criterion. Ultimately, however, the total amount of hydrogen present in the system of the methanol synthesis apparatus is an essential criterion for open-loop/closed-loop control between the two operating modes, although this is also dependent on the synthesis gas, which may vary according to the primary material quality. And since this is also affected by the hydrogen content in the synthesis gas(es), it is alternatively also possible to use this parameter as a criterion for the switchover.

In addition, it is advantageous here when the switchover between the operating modes or the establishment of the ratio of the operating modes takes place in an automated manner depending on the power available for electrolysis or the volume of hydrogen available, and the C/H ratio of the synthesis gas/biogas, so as to result in an autonomous system in which no human controller has to intervene regularly. Thus, the system is preferably set up such that it can switch between the operating modes (preferably in an automated manner) depending on the power available for the electrolysis or the volume of hydrogen available.

It is also advantageous when, in the main operating mode and the secondary operating mode, a biogas is produced from biomass and the synthesis gas is separated from the biogas in a hydrogen separator and comprises hydrogen H2 and in particular also carbon dioxide CO₂. This describes by way of example how the synthesis gas can be recovered from the biomass. A hydrogen separator comprises essentially a membrane unit that separates hydrogen (with or without CO₂) from a gas stream. As well as membrane solutions, other technologies are also conceivable here, for example pressure swing adsorption or the like.

In addition, it is considered to be advantageous that process heat which is generated by the generator and/or the oxyfuel burner and from the methanol synthesis is passed to a HyGas generator for generation of the biogas and/or to the reformer and/or to the methanol distillation or synthesis. This is advantageous since the components that require heat are supplied directly in this way with the process heat formed.

In one development, a storage means for oxygen is provided, wherein, in the main operating mode, the storage means is filled with electrolytically recovered oxygen, and the oxygen is used in the secondary operating mode for operation of the oxyfuel burner. The oxyfuel burner generally has to be operated with pure oxygen. In this way, it is possible even during secondary operation, i.e. in phases without generation of electrolysis oxygen, to effectively use the oxyfuel burner, thus resulting in 24-hour cycles over which the oxygen can be iteratively stored and provided for secondary operation.

In one development, a storage means for CO₂ is provided, wherein, in the secondary operating mode, the storage means can be filled with CO₂ recovered from the synthesis gas, and the stored carbon dioxide can be utilized in the main operating mode for methanol synthesis. In this way, in phases with high availability of green power (for instance under intense insolation/in high winds), elevated production of hydrogen can be implemented during main operation. This also gives rise to 24-hour cycles over which the carbon dioxide can be iteratively stored and provided for main operation.

In an alternative or additional development, a portion of the green power may be used to produce additional CO₂ by separation from ambient air (direct air capture-DAC) by means of a separation apparatus and to add it to the synthesis gas. In this way, in surplus H2 phases, i.e. phases with high availability of green power, an elevated yield of methanol may be achieved.

In addition, in one development, an H2 storage means may be provided, in order to generate further flexibility in H2 supply to the methanol synthesis in secondary operation as well.

Here too, a system for production of methanol is considered, having a HyGas generator for feeding a hydrogen separator set up to separate out hydrogen H2 in particular, which is fed as synthesis gas to a methanol synthesis apparatus, wherein remaining fractions from the hydrogen separator may be fed as a tail gas to an oxyfuel burner and/or a reformer and/or a generator, wherein the system is set up, in a main operating mode, to feed electrolysis hydrogen to the methanol synthesis apparatus. And no electrolysis takes place in a secondary operating mode. In addition, a controller is provided, in order to start or to increase a tail gas stream from the hydrogen separator to a generator in the event of a reduction in the electrolysis hydrogen supplied, by correspondingly reducing a tail gas stream from the hydrogen separator to the reformer and in particular also the oxyfuel burner. The electrolysis hydrogen is preferably obtained by electrolysis with power that has been generated by solar or wind energy. The hydrocarbon flow from the hydrogen separator to both the oxyfuel burner and the reformer takes place in a defined ratio which is adjusted or controlled such that a very substantially pure mixture of H2, CO and CO₂ is passed from the reformer to the methanol synthesis apparatus, which is additionally stoichiometrically suitable for the synthesis. The system is set up to execute the process according to the claims.

Further areas of applicability will become apparent from the description provided herein. It should be understood that the description and specific examples are intended for purposes of illustration only and are not intended to limit the scope of the present disclosure.

### DRAWINGS

In order that the disclosure may be well understood, there will now be described various forms thereof, given by way of example, reference being made to the accompanying drawings, in which:

FIG. 1 is a flow diagram of the system in a first working example of the main operating mode, and this plan is also used in the case of mixed forms of the operating modes;

FIG. 2 is a flow diagram of the system in a first working example of a secondary operating mode;

FIG. 3 is a flow diagram of the system in a second working example of the main operating mode, and this plan is also used in the case of mixed forms of the operating modes; and

FIG. 4 is a flow diagram of the system in a second working example of the secondary operating mode.

The drawings described herein are for illustration purposes only and are not intended to limit the scope of the present disclosure in any way.

### DETAILED DESCRIPTION

The following description is merely exemplary in nature and is not intended to limit the present disclosure, application, or uses. It should be understood that throughout the drawings, corresponding reference numerals indicate like or corresponding parts and features.

The description of the preferred embodiments of a system for methanol synthesis that follows describes a main operating mode and a secondary operating mode which differ in particular as to whether the apparatus is being supplied with sufficient green power for operation of an electrolysis apparatus, and of their individual components themselves. Green power is especially power which is obtained by photovoltaics or wind power. Since photovoltaics work only during the day, the main operating mode can alternatively also be referred to as “daytime operation”, and the secondary operating mode consequently as “nighttime operation”.

A fundamental distinction is thus made between two operating modes of the apparatus. Specifically, sufficient (renewable) power is supplied in the main operating mode to enable maximum methanol synthesis. In this operating mode, in principle, this maximum methanol synthesis is limited by the carbon content and/or oxygen content in a biogas 15 described hereinafter. In the secondary operating mode, the plant is not supplied with power generated by renewable techniques. Since it is preferable that no nonrenewable power be used, the system is throttled in the secondary operating mode such that methanol production is reduced and, instead, a significant proportion of the biogas 15 generates power in a generator 50, for operation of the individual components of the system. The term “generator” is an umbrella term for a multitude of technical means of obtaining power from organic substances. In particular, the organic substances may be burnt in a combustion chamber, resulting in evaporation of a liquid and hence generation of the desired power in a turbine.

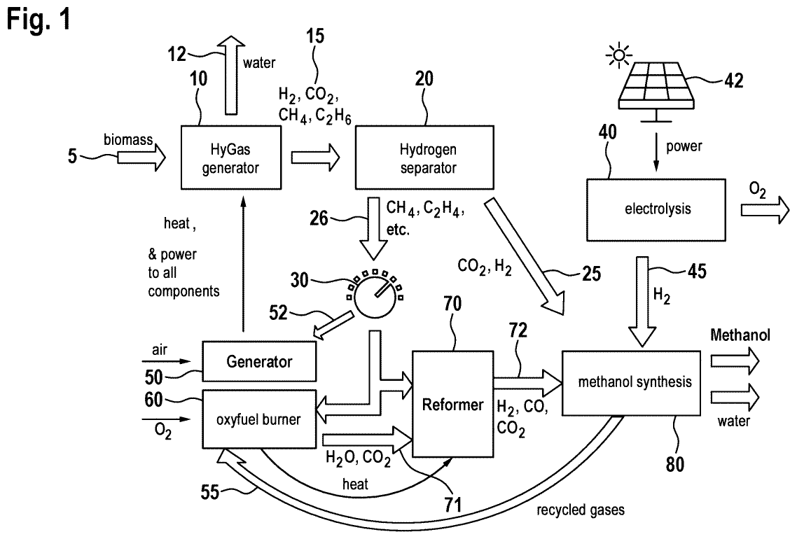

Top left in the diagram of FIG. 1, a HyGas generator 10 is supplied with biomass 5. The term “biomass” is considered to be an umbrella term and includes, for example, sewage sludge, but in particular also organic wastes, for example biowaste such as green waste or pomace, fermentation residues from biogas plants and liquid manure, but also wastes from the food industry. Owing to the German Sewage Sludge Ordinance, in particular, it is no longer permissible to burn sewage sludge. Instead, it is actually advisable to upgrade the primary materials present (or recoverable) so as to create maximum additional value, as accomplished in the methanol recovery described hereinafter. HyGas generators 10 refer in particular to devices that treat a wet organic mass with supercritical water at a pressure of more than 250 bar and a temperature of above 600° C. This splits off biogas 15, which is a gas having a high methane (CH₄) content and hydrogen (H₂) content. Also present are further (preferably short-chain) hydrocarbons, such as, in particular, C₂H₆, but also CO, CO₂. A by-product generated by the HyGas generator 10 is water 12, but this is not considered further here aside from partial utilization in the electrolysis. In particular or alternatively, the HyGas generator 10 may be an anaerobic fermenter.

The biogas 15 produced is fed to a hydrogen separator 20. Constituents usable directly in the methanol synthesis apparatus 80 are separated off therein and fed as synthesis gas 25 to the methanol synthesis apparatus 80. The synthesis gas 25 is in particular hydrogen H₂. For technical reasons, a significant proportion of carbon dioxide CO₂ may also be present. And, as the case may be, other gases may also be present in certain small amounts.

The fractions of the biogas 15 that cannot be passed onward as synthesis gas 25 are referred to hereinafter as tail gas 26 and are passed to a controller or switch 30. This is referred to hereinafter simply as “controller”. For the purposes of control technology, this may either be an open-loop controller or a closed-loop control circuit. This is an open-loop controller particularly when the available green power is being used as manipulated variable. If, by contrast, the total amount of hydrogen which is fed to the methanol synthesis apparatus 80 is used as manipulated variable, the system used may be a closed-loop control since the amount of hydrogen that is fed to the methanol synthesis apparatus 80 via a reformer 70 varies depending on the closed-loop control setting. The controller 30 divides the residual gas (mass) flow rate 26 supplied into two partial (mass) flows, where the division is different in the operating modes.

In the main operating mode, the system comprises an electrolysis apparatus 40 which is preferably supplied with renewably generated power (photovoltaic, wind power) and in which water is split into electrolysis hydrogen 45 and electrolysis oxygen. The electrolysis hydrogen 45 is passed directly to the methanol synthesis apparatus 80. The electrolysis oxygen is fed as required to an oxyfuel burner 60 or discharged from the system. The methanol synthesis apparatus 80 produces the desired biomethanol and water as a waste product. There also remain tail gases 55 that cannot be used here and are fed to a generator 50 in a recycle stream. The generator 50 burns the gases supplied thereto and thereby generates power and heat, which are in turn each fed to different components of the system where there is a corresponding demand.

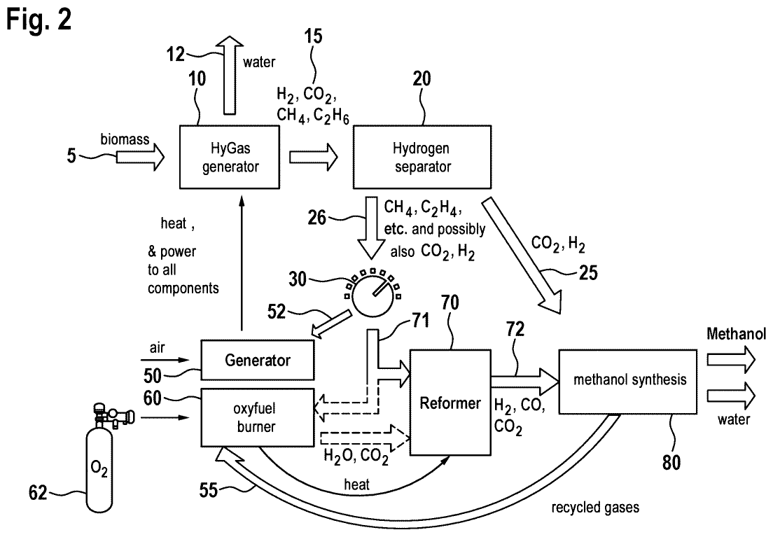

As already mentioned, the tail gas 26 fed in is divided into partial (mass) flows in a particular ratio in the controller 30 in accordance with its closed-loop control setting. The width of the arrows in FIGS. 1 and 2 schematically expresses the division ratio in the main and secondary operating modes. Accordingly, in the main operating mode of FIG. 1, a greater proportion (in particular, optionally, completely to an extent of 100%) goes into a processing operation comprising an oxyfuel burner 60 and a reformer 70. A corresponding mass flow leads from the processing operation 60, 70 as a further synthesis gas 72 to the methanol synthesis 80. The tail gas 26 which is fed to the processing operation 60, 70 is divided in a particular ratio between the oxyfuel burner 60 and the reformer 70 within the processing operation 60, 70. This ratio is determined so as to enable a stoichiometrically best possible synthesis of the methanol in the methanol synthesis apparatus 80. This ratio can be determined in an automated manner by means of a closed-loop control circuit (not shown).

The oxyfuel burner 60 is operated with pure oxygen, but substoichiometrically, and the output products therefrom are especially water and CO₂, which are passed to the reformer 70. In addition, the reformer 70 contains a proportion of the tail gas 26, and transformation processes take place therein. These endothermic processes are supplied with heat from the oxyfuel burner 60 and/or the generator 50. The product gases from the reformer 70 are H₂, CO and CO₂, and are fed as further synthesis gas 72 to the methanol synthesis 80.

FIG. 2 shows the secondary operating mode in which no hydrogen is produced from electrolysis and provided to the methanol synthesis apparatus 80. Since this reduces the production of methanol in the methanol synthesis apparatus 80, there is also a drop in demand for the synthesis gases 25 and 72, with a particular reduction in the CO₂ demand. Thus, in particular, a portion of the synthesis gas 25 separated (or separable) in the hydrogen separator 20 is not fed to the methanol synthesis apparatus 80 but introduced into the stream of tail gas 26. In other words, preferably only a reduced flow of the synthesis gas 25 is separated from the biogas 15. The respective proportion results from the demand in the methanol synthesis apparatus 80.

In the secondary operating mode, a greater proportion of the tail gas 26 optionally thus modified is passed as generator gas 52 to the generator 50, as indicated by the width of the arrow in FIG. 2. It is combusted therein in order thereby to generate electrical power and heat, which is consumed in the components of the system having a corresponding demand. In this way, the demand for power not obtained by renewable means for the overall system of the methanol synthesis can be reduced or avoided. The heat that arises is also passed to the corresponding components of the system, in a comparable manner to the oxyfuel burner 60.

Depending on the specific gas compositions, it may be possible for the proportion of the tail gas 26 that is to be processed to be merely passed to the reformer 70, such that the oxyfuel burner 60 in this case is not required. It is also alternatively possible for the proportion that is passed to the processing operation 60, 70 to be set to zero. The latter case occurs when the volume flow rate of the synthesis gas 25 is sufficient for the methanol synthesis apparatus 80. Fractions of the synthesis gas 25 that are not used for the synthesis are fed to the generator 50 as recycled gases 55 and utilized thermally.

The controller 30 may thus feed 100% of the tail gas 26 to the generator 50. On the other hand, in the secondary operating mode, there is no state in which 100% of the tail gas 26 is fed to the processing operation 60, 70. This state, i.e. that in which the generator 50 is not supplied with tail gas 26, is viable in the main operating mode if at all. In the main operating mode too, the generator 50 is nevertheless always used since the recycled gases 55 are utilized thermally therein. Intermediate stages can also be established (preferably with infinite adjustability). As an alternative to intermediate stages, a respective complete switchover of the modes of operation is also possible.

It is also optionally possible to use an oxygen storage means 62. If the system is operated with photovoltaic power in the main operating mode during the day, a surplus of electrolysis oxygen is typically obtained. This surplus is found as the difference between the oxygen obtained electrolytically and the oxygen demand of the oxyfuel burner 60. This surplus can be stored in the oxygen storage means 62 for the secondary operating mode during the night, such that the oxyfuel burner 60 is also able to work at night. This is because the oxyfuel burner 60 has to be operated with pure oxygen in order to assure its function and in order to ensure that no extrinsic gases, for example nitrogen, are fed to the methanol synthesis apparatus 80.

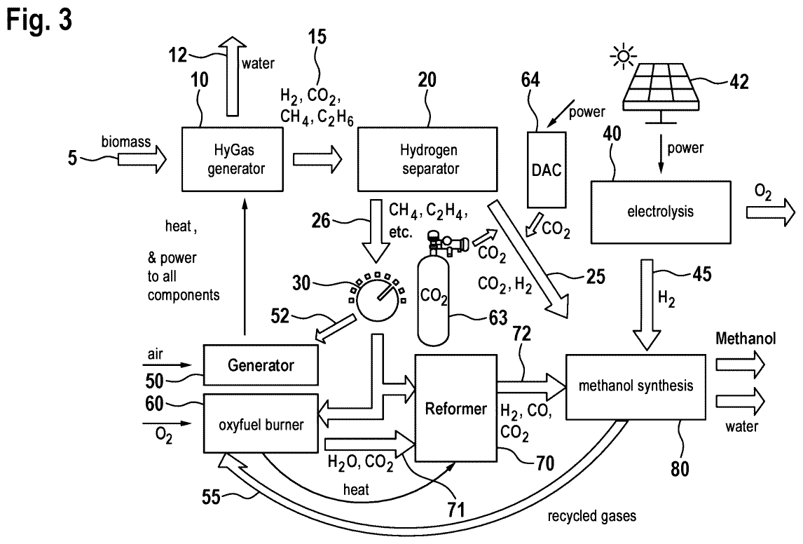

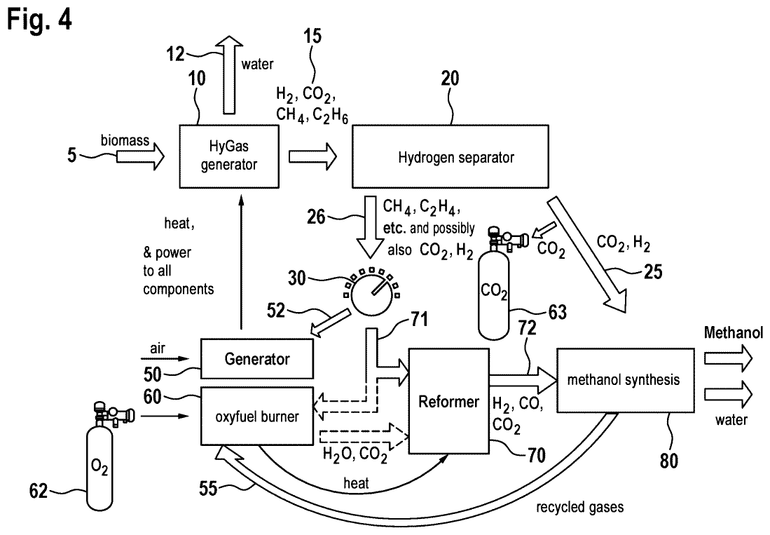

FIGS. 3 and 4 each show a second working example of a flow diagram of the main and secondary operating modes. This working example differs only in one aspect from the flow diagram shown in FIGS. 1 and 2, and only this aspect will be addressed hereinafter. In the further working example of the flow diagram, with high availability of green power, i.e., for example, under intense insolation, a portion of the power is used in a separation apparatus 64 to separate carbon dioxide out of the ambient air (direct air capture) and to add it to the synthesis gas 25. In this way, it is possible in phases of surplus green power to prevent an excess of hydrogen from arising in the methanol synthesis apparatus 80. Instead, the excess green power can be utilized to produce further carbon dioxide, in order to maximize the methanol yield.

Alternatively or additionally, it is also possible to use a CO₂ storage means 63, which is filled in secondary operation with surplus carbon dioxide, which can be added again to the synthesis gas 25 during main operation, in phases of surplus green power. In this case, the controller 30 controls the filling and emptying of the CO₂ storage means 63 and the supply of green power to the separation apparatus 64.

Unless otherwise expressly indicated herein, all numerical values indicating mechanical/thermal properties, compositional percentages, dimensions and/or tolerances, or other characteristics are to be understood as modified by the word “about” or “approximately” in describing the scope of the present disclosure. This modification is desired for various reasons including industrial practice, material, manufacturing, and assembly tolerances, and testing capability.

As used herein, the phrase at least one of A, B, and C should be construed to mean a logical (A OR B OR C), using a non-exclusive logical OR, and should not be construed to mean “at least one of A, at least one of B, and at least one of C.”

The description of the disclosure is merely exemplary in nature and, thus, variations that do not depart from the substance of the disclosure are intended to be within the scope of the disclosure. Such variations are not to be regarded as a departure from the spirit and scope of the disclosure.

What is claimed is:

1. A process for producing methanol, comprising:
feeding a synthesis gas that has been recovered from a biomass to a methanol synthesis apparatus,
wherein, in a main operating mode in which sufficient electrical power is available for electrolytic hydrogen recovery, correspondingly electrolytically recovered hydrogen is fed to the methanol synthesis apparatus, and
wherein, in a secondary operating mode in which insufficient electrical power is available for electrolytic production of hydrogen, a tail gas that arises from a biogas recovered from the biomass on removal of the synthesis gas is fed to a generator in order to provide electrical power for apparatuses involved in the process.

2. The process as claimed in claim 1, wherein, at least in the main operating mode, and preferably also in the secondary operating mode, the tail gas is fed to a processing operation in order to produce a further synthesis gas which is also fed to the methanol synthesis apparatus.

3. The process as claimed in claim 2, wherein, in the processing operation, the tail gas is fed in a particular ratio to an oxyfuel burner and a reformer, wherein an oxidation is conducted in the oxyfuel burner and the oxidized gas is fed to the reformer, and the reformer conducts a reduction to supply the further synthesis gas to the methanol synthesis apparatus.

4. The process as claimed in claim 1, further comprising switching between the main operating mode and the secondary operating mode depending on available green power or the available hydrogen, or selecting mixed forms of the main operating mode and the secondary operating mode,
wherein the mixed forms differ in a ratio of the tail gas that is fed either to the generator or to the processing operation.

5. The process as claimed in claim 4, wherein the tail gas is divided into two substreams that are passed to the generator or the processing operation.

6. The process as claimed in claim 5, wherein the switchover or division takes place in an automated manner depending on a current available for electrolysis or an amount of the hydrogen from the methanol synthesis apparatus.

7. The process as claimed in claim 1, wherein, in the main operating mode and the secondary operating mode, the biogas is produced from the biomass and the synthesis gas is separated from the biogas in a hydrogen separator and comprises hydrogen H₂ and in particular also carbon dioxide CO₂.

8. The process as claimed in claim 3, wherein process heat which is generated by the generator and/or the oxyfuel burner is passed to a HyGas generator for generation of the biogas and/or to the reformer.

9. The process as claimed in claim 3, further comprising providing a storage means for oxygen,
wherein, in the main operating mode, the storage means is filled with electrolytically recovered oxygen, and the oxygen is used in the secondary operating mode for operation of the oxyfuel burner.

10. The process as claimed in claim 1, further comprising providing a storage means for CO₂,
wherein, in the secondary operating mode, the storage means can be filled with CO₂ recovered from the synthesis gas, and the CO₂ stored in the storage means can be utilized in the main operating mode for methanol synthesis.

11. The process as claimed in claim 1, wherein a portion of green power is used to produce additional CO₂ by separation from ambient air (direct air capture-DAC) by means of a separation apparatus and to add it to the synthesis gas.

12. A system for production of methanol, the system comprising:
a hydrogen separator configured to separate hydrogen;
a HyGas generator for feeding a biogas to the hydrogen separator;
a methanol synthesis apparatus, the hydrogen from the hydrogen separator being feedable as synthesis gas to the methanol synthesis apparatus, remaining fractions from the hydrogen separator being fed as a tail gas to at least one of an oxyfuel burner, a reformer, and a generator, wherein the system is configured to feed electrolysis hydrogen to the methanol synthesis apparatus in a main operating mode and to switch to a secondary operating mode in which no electrolysis hydrogen is supplied; and
a controller configured to start or to increase a tail gas stream from the hydrogen separator to a generator in the event of a reduction in the hydrogen fed to the methanol synthesis apparatus, especially the electrolysis hydrogen, by correspondingly reducing the tail gas stream from the hydrogen separator to the at least one of the oxyfuel burner and the reformer.
